(19) United States
(12) Patent Application Publication
(10) Pub. No.: US 2002/0172967 A1
(43) Pub. Date: Nov. 21, 2002

(54) Identification of non-covalent complexes by mass spectrometry

(76) Inventors: Thomas R. Gadek, Oakland, CA (US); Denise Ockey, Oakland, CA (US)

Correspondence Address: Supervisor, Patent Prosecution Services, PIPER MARBURY RUDNICK & WOLFE LLP, 1200 Nineteenth Street, N.W., Washington, DC, 20036-2412, US

(21) Appl. No.: 10/073,077

(22) Filed: Feb. 12, 2002

Related U.S. Application Data
(60) Provisional application No. 60/268,556, filed on Feb. 13, 2001.

(51) Int. Cl. C12Q 1/68

(52) U.S. Cl. 435/6

(57) ABSTRACT

Methods for identifying novel drug leads or binding compounds that have an affinity for a target molecule involving screening known drug fragment molecules and derivatives thereof, preferably using mass spectrometry are disclosed.

This application claims priority from U.S. Provisional Application Serial No. 60/268,556 filed Feb. 13, 2001. The entirety of that provisional application is incorporated herein by reference.

### BACKGROUND OF THE INVENTION

1. Field of the Invention

The present invention generally relates to methods for identifying drug lead and drug compounds from libraries of compounds. The method involves the use of mass spectrometry to observe the existence of a noncovalent complex between a target biomolecule and a small molecule ligand, the determination of the relative concentration of the free and complexed target biomolecule and the equilibrium binding dissociation constant for the complex as an aid in the identification and design of ligands that bind to a target biomolecule as a 1:1 noncovalent complex.

2. Discussion of the Background

Traditionally, new drug lead discovery follows a route that involves the synthesis and/or isolation of a compound followed by its evaluation through a series of assays. A first step in the characterization of a new drug often involves identification of molecules that display a high binding affinity to a target biomolecule.

One tool for discovering new drug lead compounds is random screening of synthetic chemical and natural product libraries to determine compounds that bind to a particular target molecule (i.e., the identification of ligands of that target). Using this method, ligands may be identified by their ability to form a physical association with a target molecule or by their ability to alter a function of a target molecule.

When physical binding is sought, a target molecule is typically exposed to one or more compounds suspected of being ligands and assays are performed to indicate the presence of complexes between the target molecule and one or more of those compounds are formed. Such assays are well known in the art and test for gross changes in the target molecule (e.g., changes in size, charge, function, or mobility) that indicate complex formation.

Where functional changes are measured, assay conditions are established that allow for measurement of a biological or chemical event related to the target molecule (e.g., enzyme-catalyzed reaction or receptor-mediated enzyme activation). To identify an effect, the function of the target molecule is determined before and after exposure to the test compounds.

Existing physical and functional assays have been used successfully to identify new drug leads for use in designing therapeutic compounds. There are, however, limitations inherent to those assays that compromise their accuracy, reliability and efficiency.

A major shortcoming of existing assays relates to the problem of “false positives”. In a typical functional assay, a “false positive” is a compound that triggers a positive response in the assay but which compound is either incapable of associating with the target biomolecule or is not effective in eliciting the desired physiological response in live cells or in an organism. In a typical physical assay, a “false positive” is a compound that, for example, attaches itself to the target but in a non-specific manner (e.g., non-specific binding where the ratio of ligand to target biomolecule in the bound state is frequently greater than 1:1). False positives are particularly prevalent and problematic when screening higher concentrations of putative ligands because many compounds have non-specific affects at those concentrations.

In a similar fashion, existing assays are plagued by the problem of “false negatives”, which result when a compound gives a negative response in the assay, but which compound is actually a ligand for the target. False negatives typically occur in assays that use concentrations of test compounds that are either too high (resulting in toxicity) or too low relative to the binding or dissociation constant of the compound to the target.

Another major shortcoming of existing assays is the limited amount of information provided by the assay itself. While the assays may correctly identify compounds that attach to or elicit a response from the target molecule, they typically do not provide any information about either specific binding site on the target molecule or structure activity relationships between the compound being tested and the target molecule. The inability to provide such information is particularly problematic where large numbers of compounds are subjected to the screening assay used to identify lead compounds for further study.

It has recently been suggested that x-ray crystallography can be used to identify the binding sites of small organic molecules (e.g. solvents) on macromolecules. However, this method cannot determine the relative binding affinities at different sites on the target. Moreover, this approach is not a useful high throughput screening method for rapidly testing many compounds that are chemically diverse, but it is limited to mapping the binding sites of only a few organic molecules due to the long time needed to determine individual crystal structures.

In response to an increasing demand for novel compounds useful in the effective treatment of various maladies, the medical research community has developed a number of strategies for discovering and optimizing new therapeutic drugs. For the most part, these strategies depend upon molecular techniques that allow the identification of ligands capable of tightly binding to a given target biomolecule. Once identified, these ligands may then carry out their therapeutic functions by activating, inhibiting or otherwise altering the activity of the molecular target to which they bind.

In one such strategy, new therapeutic drugs are identified by screening combinatorial libraries of synthetic small molecule compounds, determining which compound(s) have the highest probability of providing an effective therapeutic and then optimizing the therapeutic properties of the identified small molecule compound(s) by synthesizing structurally related analogs and analyzing them for binding to the target molecule (Gallop et al (1994)). However, this process is not only time consuming and costly, but it often does not provide for the successful identification of a small molecule compound having sufficient therapeutic potency for the desired application. For example, while the preparation and evaluation of combinatorial libraries of small molecules has proven somewhat useful for new drug discovery, the identification of small molecules for difficult molecular targets (e.g., such as those useful for blocking or otherwise taking part in protein-protein interactions) has not been particularly effective (Brown (1996)).

One issue that limits the success of combinatorial library approaches is that it is possible to synthesize only a very small fraction of the possible number of small molecules. For example, greater than 10⁶⁰ different small molecules having valid chemical structures and molecular weights under 600 daltons can be envisioned. However, even the most ambitious of small molecule combinatorial library efforts have been able to generate libraries of only tens to hundreds of millions of different compounds for testing. In this regard, a library of one billion molecules would represent 0.000000000000000000000000000000000000000000000001% of the estimated total of 10⁶⁰ small molecules. Therefore, combinatorial technology allows one to test only a very small subset of the possible small molecules, thereby resulting in a high probability nearing certainty that the most potent small molecule compounds will be missed. Thus, suitable small molecule compounds having the required availability, activity or chemical and/or structural properties often cannot be found. Moreover, even when such small molecule compounds are available, optimization of those compounds to identify an effective therapeutic often requires the synthesis of an extremely large number of structural analogs and/or prior knowledge of the structure of the molecular target for that compound. Furthermore, screening large combinatorial libraries of potential binding compounds to identify a lead compound for optimization can be difficult and time-consuming because each and every member of the library must be tested. It is evident, therefore, that novel methods for rapidly and efficiently identifying new small molecule drug leads are needed.

Two screening techniques have been mainly used to identify tight binding ligands: (1) screening and recombining extremely large populations of compounds, and (2) performing multiple rounds of screening and recombination on relatively small populations, where additional building blocks are gradually introduced. In this second approach, many rounds of selection, recombination and building block introduction are required to identify the optimal building block recombinations. The first of these methods suffers from the same limitation as the combinatorial library, where it is not feasible to have a library large enough to contain even a single molecule of each of the 10⁶⁰ different small molecules having valid chemical structures and molecular weights under 600 daltons which can be envisioned. The second method reduces the number of compounds to be made and tested by starting with smaller molecular weight building blocks that are covalently combined, selected and recombined in different molecular arrangements. While this method may be able to identify individual building blocks that can bind to the target biomolecule, it suffers from a need to consider a very large number of possible covalent connectivities in the combination and recombination step. These connectivities control the orientation of the building blocks relative to each other and as such, control the three dimensional presentation of the combined building blocks to the target biomolecule. Consequently, as the number of building blocks for combination and recombination increase, the number of compound combinations and/or the number of iterations of combination, selection and recombination limit the practicality of the approach.

According to more recent methods, compounds are screened to identify lead compounds that can be used in the design of new drugs that alter the function of the target biomolecule. These new drugs can be structural analogs of identified lead compounds or can be conjugates of one or more of such lead compounds. Because of the problems inherent to existing screening methods, the methods are often of little help in designing new drugs.

One such method involves the study of several sets of ligands where the members of each set are defined by their ability to compete with each other for one of several potential binding sites on a target biomolecule. Thus, the target biomolecule is capable of using more than one binding site to simultaneously bind a single member of more than one set of ligands. Simultaneous interactions of small ligands with proteins often have unique collective properties that are different than any single constituent. A recently reported approach for identifying high affinity ligands for molecular targets of interest is by determining structure-activity relationships (SAR) from nuclear magnetic resonance (NMR) analysis, i.e., “SAR by NMR” (Shuker et al (1996) and U.S. Pat. Nos. 5,698,401 and 5,989,827). In this approach, NMR determines the physical structure of a target protein and small molecule building blocks that bind to the protein in proximity to each other on the protein surface are identified. Small molecules which are bound simultaneously or individually to the target protein with proximity and well defined relative orientation are then coupled together with a linker that maintains or enforces the proximity and relative orientation in order to obtain compounds that bind to the target protein with higher affinity than the unlinked compounds alone. Thus, by having available the NMR structure of the target protein, the lengths of linkers for coupling two adjacently binding small molecules can be determined and small molecule ligands can be designed. This approach has been useful for identifying compounds that bind to FK506 binding protein with a K_{d}=20 nM (Shuker et al, supra) and to stromelysin with a K_{d}=15 nM (Hajduk et al (1997)).

However, while the SAR by NMR method is powerful, it also has serious limitations. For example, the approach requires large amounts of target protein (>200 mg) and the protein typically must be ¹⁵N-labeled so that it is useful for NMR studies. Moreover, the SAR by NMR approach usually requires that the target protein be soluble to >0.3 mM and have a molecular weight less than about 25-30 kDa. Additionally, the structure of the target protein is first solved by NMR, a process that often can require a 6 to 12 month time commitment.

WO 99/49314 discloses a method, in which a population of small molecules are “pre-selected” for the ability to bind to a molecular target, where the small molecules that bind with the highest affinity are then chemically linked in various combinations to provide a library of potential high affinity binding ligands. The library of potential binding ligands is then screened using techniques such as ELISA for the presence of one or more compounds that bind to the target molecule with very high affinity.

WO 00/00823 discloses a method for identifying small organic molecule ligands for binding to biological target molecules. The method involves obtaining a biological target molecule that contains a chemically reactive group, combining the biological target molecule with one or more members of a library of organic compounds that are capable of covalently bonding to the chemically reactive group, and identifying the organic compound(s) that forms a covalent bond with the chemically reactive group. The reference discloses that methods, including mass spectrometry, liquid chromatography, NMR, capillary electrophoresis and x-ray crystallography, may be used to identify the organic compound. WO 00/00823 requires the use of organic compound(s) that have already demonstrated an ability to bond with the biological target molecule.

U.S. Pat. No. 6,335,155 discloses a method for identifying small organic molecule ligands that are capable of binding to selected sites on biological target molecules of interest. In the disclosed process, small molecular ligands are screened by forming a covalent bond between chemically reactive groups in the ligand and on the target.

There are limited reports in the literature that use mass spectrometry for the detection of non-covalent interactions between macrolides and proteins. For example, non-covalent interactions between FKBP with FK506 and Rapamycin have been determined using ion-spray mass spectrometry. Ganen et al (1991). Peak integration was used to determine relative affinities of the two macrolides for FKBP. The relative affinities agreed with their known absolute affinities.

Gao et al (1996) discloses the use of ESI-MS to identify amino acid residues that maximize the binding affinities by secondary interactions with the active site of CAII. Gao et al discloses that this approach to study non-covalent complexes permits increased performance that results from analyzing free ligands generated from the dissociation of complexes (rather than analyzing intact complexes).

Cheng et al (1995) discloses the use of ESI-MS to characterize non-covalent complexes of proteins with mixtures of ligands. In particular, Cheng et al discloses the study of non-competitive binding of inhibitors derived from para-substituted benzenesulfonamides to bovine carbonic anhydrase.

Jorgensen et al (1998) discloses that solution-binding constants for complexes between glycopeptide antibiotics and several peptide ligands can be determined directly from a single measurement by ESI-MS.

Matrix metalloproteinase enzymes are a family of proteinases that include collagenases, gelatinases and stromelysins. These enzymes appear to be involved in connective tissue degradation and have been implicated in such diseases as arthritis and cancer. This enzyme family requires zinc and calcium for activity. The zinc- and calcium-binding stoichiometry for stromelysin, a member of this family has been measured by ESI-MS (Hu et al (1994)).

From the above, it is evident that there is a need for novel techniques useful for rapidly and efficiently identifying small molecule drug lead compounds that are capable of binding with high affinity to a molecular target of interest.

### SUMMARY OF THE INVENTION

The present invention provides a rapid and efficient screening method for identifying ligands that bind to therapeutic target molecules. The present inventors have discovered a method for rapidly and efficiently identifying sets of molecules that are capable of binding to unique sites on a target with measurable affinity, where the identified sets of molecules are useful, for example, in preparing drug lead compounds. The method of the invention allows one to assay only the most favorable compounds for binding to a target biological molecule without the need for screening all possible small molecule compounds and combinations thereof for binding to the target, as is required in standard combinatorial library approaches. The method of the invention also allows one to study the competition of target binding ligands with a parent molecule (drug lead or drug molecule) for binding to a target. The association of a target biomolecule with libraries of molecules whose individual members may or may not be known to associate with the target can be studied by mass spectrometry in order to identify or confirm those molecules which bind to the target biomolecule. This can be accomplished by studying the association of the target biomolecule with individual molecules or mixtures of molecules selected from libraries of compounds whose molecular weight and chemical structures are known and which may or may not be known to contain compounds which bind to the target biomolecule.

With regard to the above, in one aspect, the present invention is directed to a method (1) for identifying compounds that bind to a target of interest, by (a) assembling a first set of target binding ligands that compete for binding to a first binding site on the target;

(b) assembling a second set of target binding ligands that compete for binding to a second binding site on the target;

(c) chemically linking at least one member of the first set and at least one member of the second set to provide a first set of linked ligands; and

(d) screening the set of linked ligands to identify members thereof that bind to the target.

Other aspects of the invention include the method (1) above, where:

(2) assembling step (a) or assembling step (b) comprises measuring binding of target binding ligands to the target by mass spectroscopy;

(3) target binding ligands having a disassociation constant, K_{d}, equal to 500 μM or less are assembled into a set;

(4) the identified linked ligands have a disassociation constant, K_{d},equal to 500 μM or less, preferably 100 μM or less;

(5) the first binding site is the same as the second binding site;

(6) the first binding site is not the same as the second binding site;

(7) assembling step (b) comprises determining binding of target binding ligands to the target having at least one member of the first set of target binding ligands bound thereto;

(8) the target is a target biomolecule;

(9) the target biomolecule is a polypeptide, protein, DNA, RNA or polysaccharide;

(10) step (c) comprises forming a covalent bond or attachment linking the member of the first set and the member of the second set;

(11) screening of step (d) comprises a biological measurement;

(12) a member of the first set and a member of the second set bind to the target in a 1:1 ratio;

(13) the method of (1) further comprises assembling a third set of target binding ligands that compete for binding to the first binding site on the target and a fourth set of target binding ligands that compete for binding to the first binding site on the target, where members of each of the third set and the fourth set compete with members of the first set for binding to the first binding site, but members of the third set do not compete with members of the fourth set for binding to the target.

(14) the method of (13) further comprises covalently linking at least one member of the third set or the fourth set and at least one member of the second set to provide a second set of linked ligands; and screening the second set of linked ligands to identify members thereof that bind to the target.

The invention is also directed to the following:

(15) A method of preparing a drug lead compound that binds to a target, comprising

covalently linking at least one member of a first set of target binding ligands that compete for binding to a first binding site on the target and at least one member a second set of target binding ligands that compete for binding to a second binding site on the target set to provide a first set of linked ligands.

The method (15) above where:

(16) the first binding site is the same as the second binding site;

(17) the first binding site is not the same as the second binding site;

(18) the method (15) further comprises screening the set of linked ligands to identify members thereof that bind to the target;

(19) the method (15) further comprises covalently linking at least one member of a third set of target binding ligands that compete for binding to the first binding site on the target or at least one member of a fourth set of target binding ligands that compete for binding to the first binding site on the target to form a second set of linked ligands, where members of each of the third set and the fourth set compete with members of the first set for binding to the first binding site, but members of the third set do not compete with members of the fourth set for binding to the target.

The invention is also directed to the following:

(20) a method for inhibiting the binding of a second biomolecule to a first biomolecule, comprising:

contacting the first and second biomolecules with a binding inhibitory amount of a compound identified according to any of methods (1)-(19) above, where the compound binds to the first biomolecule and inhibits the binding of the second biomolecule.

These and other aspects of the invention are described in more detail and will become apparent from the detailed description below.

### BRIEF DESCRIPTION OF THE DRAWINGS

FIG. 1 is an example of a typical mass spectrum of ligands noncovalently bound to a protein. Peaks corresponding to each of two ligands (acetohydroxamic acid; 4-(4′-cyanophenyl) phenol) that are noncovalently bound to the protein are observed as well as peaks corresponding to the ternary complex of the two ligands bound to different binding sites on the protein (stromelysin). This is a demonstration of two ligands from two sets of target binding ligands that do not compete for the same binding site on a protein (and define two distinct binding sites).

FIG. 2 is a reconstructed mass spectrum of a competition experiment between 4-phenylpyridine and 4-methoxy-N-phenylbenzamide for binding to stromelysin. Masses corresponding to the noncovalent complex of each of these ligands complexed to the protein separately are observed. The mass corresponding to these ligands bound to the protein simultaneously is absent. This is a demonstration of two ligands from the same set of target binding ligands that compete for the same binding site on a protein (and define one binding site).

FIGS. 3 is a reconstructed mass spectra of a competition between 4-phenylpyridine and N-(4-cyanophenyl)-2-phenylacetamide for binding to stromelysin. Masses corresponding to the ligands bound separately are present; however, a mass corresponding to the two ligands bound to the protein simultaneously is also present. This is a demonstration of two ligands that do not compete for the same binding site on the protein.

FIG. 4 is a graph of titration curves used to determine the IC₅₀ of the squaric acid fragment 8A and moniliformin. The IC₅₀ of moniliformin was determined to be 12 μM, and the IC₅₀ of fragment 8A was determined to be 60 μM. Fragments 8D and 8N did not have the ability to block function as determined by this method. This demonstrates the ability of the method of the invention to identify candidate target binding ligands that form specific noncovalent complexes with target biomolecules and whose ability to block function of the target biomolecule can be determined by other methods.

### DETAILED DESCRIPTION OF THE INVENTION

Definitions

“Target biomolecule” as used herein means any biological molecule of interest, for which a high affinity binding ligand is desired, for example, for use in an assay or treatment method.

“Candidate target binding ligand” as used herein means any organic compound that can be assayed for binding to a target, for example a target biomolecule. Candidate target binding ligands may be present in any library of organic compounds that is commercially available, is prepared using combinatorial chemistry methods or that can be synthesized using well know chemical methods. Suitable libraries include EST polynucleotide libraries, peptide libraries and libraries of organic compounds.

“Set of target binding ligands” means a set of compounds that bind to a target and that compete with each other for only one target binding site.

“Linked ligand” means two or more target binding ligands covalently bonded together.

“Parent molecule” or “parent” means a molecule for which can span or bridge more than one binding site on a target molecule.

“Substructural unit” means an open valence structure derived by breaking one or more bonds in a parent molecule, for example, a drug or drug lead molecule.

“Fragment molecule” means a molecule in which the open valencies of a substructural unit have been completed with radicals, for example, hydrogen or other functional groups to provide a stable compound.

Detailed Description

New compounds are continuously being synthesized through combinatorial chemistries. Such synthetic methodologies have greatly increased the number of new structurally diverse compounds that can be screened as possible new therapies in the pharmaceutical industry. Several instrumental techniques are used to study non-covalent macromolecular interactions are available such as fluorescence spectroscopy, calorimetry, ultracentrifugation, differential scanning calorimetry, nuclear magnetic resonance, and x-ray crystallography. One of the emerging technologies for the study of protein interactions is mass spectrometry and in particular, electrospray ionization mass spectrometry (ESI-MS). This technique, which does not involve the use of a chromophore or radiolabeling, is commonly used to determine the molecular masses of proteins, peptides, amino acid compositions, and amino acid sequences. It has also been used to study non-covalent interactions. The presence of a non-covalent complex can be observed directly as an ion of increased mass. In general, this method requires very little sample and is a fast technique enabling its use for high throughput screening. Non-covalent interactions of proteins with peptides, metal ions, and organic molecules such as FK506 have demonstrated the utility of this technique. Ganen et al (1991).

The present invention is based on a method involving the use of mass spectrometry for rapid, efficient, accurate, and reliable selection, identification, and design of new drug lead and new drug molecules. The method of the invention preferably uses mass spectrometry for (1) the identification of ligands that non-covalently bind to a target biomolecule from a library or libraries of candidate target binding ligands; (2) the identification of sets of target binding ligands that bind to unique sites on the target biomolecule; (3) the determination of the relative or absolute binding affinities of the target binding ligands; (4) the identification of linked ligands that bind to a target biomolecule from a library of linked molecules (linked ligands) that have been prepared by the covalent linkage of individual members of two or more sets of target binding ligands shown to bind individually to unique sites on the target biomolecule; (5) the determination of the relative or absolute binding affinities of those linked ligands; (6) the selection of enhanced ligands as those molecules that bind to the target biomolecule as a 1:1 complex; and (7) the selection of drug leads or drug molecules as those linked ligands that bind to the target biomolecule as a 1:1 complex with higher affinity (i.e lower equilibrium dissociation constant) than any of the molecules that were combined to form the linked ligand.

Information about the structure/activity relationships between target binding ligands identified by the method of the invention can be used to design new drug leads and/or drugs that are ligands to the target molecule.

There are many benefits to using mass spectrometry for lead discovery. Masses of high molecular weight compounds can be detected at mass-to-charge ratios that are easily determined by most mass spectrometers (typical m/z ranges of up to 2000 to 3000). ESI-MS, in particular, works well for charged, polar or basic compounds and for analyzing multiply charged compounds with excellent detection limits and allows the presence or absence of fragmentation to be controlled by controlling the interface lens potentials. Electrospray ionization (ESI) is also compatible with MS/MS methods.

In ESI, a dilute solution of a peptide, protein, or other biomolecule is slowly pumped through a hypodermic needle. The sample may be introduced via flow injection or LC/MS. Typical flow rates range from less than 1 microliter per minute up to about one milliliter per minute. ESI is particularly useful for large biological molecules that are otherwise difficult to vaporize or ionize. The needle is held at a high voltage and the strong electric field at the end of the needle charges the nebulized solution and creates charged droplets. The charged droplets evaporate water to ultimately yield molecular ions that travel into the vacuum chamber through a small orifice. During the process of solvent evaporation, the non-covalently bound complex is transferred from solution to gas phase. (Hu et al (1994)). Gentle desolvation conditions are generally required to maintain the intact gas-phase complex. The orifice is heated to ensure that the ions are completely desolvated. Charged droplets are emitted from a hypodermic needle and shrink as they evaporate solvent before entering a vacuum chamber. Heat and gas flows may be used to aid desolvation. ESI of proteins produce multiply charged ions with the number of charges tending to increase as the molecular weight increases. The number of charges on a given ionic species may be determined by methods such as: (1) comparing two charge states that differ by one charge and solving simultaneous equations; (2) looking for species that have the same charge but different adduct masses; and (3) examining the mass-to-charge ratios for resolved isotopic clusters. The methods of ESI and ESI-MS and parameters needed to conduct these methods are well known in the art.

Important aspects of the method for determining the affinities of molecules with targets are the relatively mild conditions of ionization and introduction into the mass spectrometer. The target protein is observed as a molecular ion generally without any associated water molecules, yet it is introduced into the spectrometer as a droplet in water at equilibrium with a parent or ligand molecule. The complex of the target biomolecule with a ligand is formed in solution and that equilibrium can be studied in the gas phase within the mass spectrometer. The soft ionization and rapid removal of water within the spectrometer allows a 1:1 noncovalent complex to be observed directly in the gas phase. Additionally, the relative concentrations of free target biomolecule and complexes of the target biomolecule with a ligand, measured as the relative intensities of the signals observed at the m/z corresponding to the mass of the complexes, are measured without apparently perturbing the equilibrium which had been established in the solution phase. ESI offers better sensitivity than conventional mass spectrometry and milder interface conditions can be used to obtain intact non-covalent complex ions with a good signal-to-noise ratio.

The gentleness of the electrospray ionization process allows intact protein complexes to be directly detected by mass spectrometry. Further, it has now been discovered that the ESI-MS observations for weakly bound gas phase systems reflect the nature of the interaction found in the condensed phase. Stoichiometry of the complex can be easily obtained from the resulting mass spectrum because the molecular weight of the complex is directly measured. For the study of protein interactions, ESI-MS is complementary to other biophysical methods, such as NMR and analytical ultracentrifugation. However, mass spectrometry offers advantages in speed, sensitivity, and stoichiometry.

The present invention provides a method of screening compounds to identify molecules that bind to a specific target molecule. As an optional first step, and prior to assembling the first and second sets of target binding ligands discussed in more detail below, candidate target binding ligands may be pre-screened to identify ligands that bind to a target, preferably a target biomolecule. The candidate ligands may be any known compounds, including compounds present in combinatorial libraries. Any in vitro assay that allows one to detect binding of the target biological molecule by an organic compound may be employed for screening the candidate target binding ligands. Suitable assays include, for example, ELISA assays, other sandwich-type binding assays, binding assays which employ labeled molecules such as radioactively or fluorescently labeled molecules, fluorescence polarization, calorimetry, protein denaturation, resistance to proteolysis, gel filtration, equilibrium dialysis, surface plasmon resonance, X-ray crystallography, and the like. Such assays measure the ability of library members to bind directly to the target. A preferred assay is the use of mass spectroscopy to determine candidate target binding ligands that bind to the target with or below a predetermined dissociation constant. The dissociation constant (Kd) is preferable less than 100 mM, more preferable less than 1 mM, and most preferably less than 1 micromolar

In the pre-screening step and in other mass spectroscopy assays described below, the formation of a noncovalent complex between a molecule and a target biomolecule can be detected by mass spectrometry for each charge state as an increase in a signal at a mass to charge ratio equal to the sum of the mass to charge ratio of the molecule plus the mass to charge ratio of the target biomolecule. The stoichiometry of the noncovalent complex can be detected by mass spectrometry for each charge state as the appearance of a signal or signals at a mass to charge ratio(s) corresponding to the sum of the mass to charge ratios of the target biomolecule and one or more molecules under study. At each charge state, the relative intensities of the signals for the biomolecule and noncovalent complexes of the molecule and biomolecule are a reflection of their relative concentration in the sample introduced into the mass spectrometer. Consequently, this intensity data can be used to calculate equilibrium binding affinities.

It is convenient to use a conventional ELISA well plate for the mass spectrometry assays, although any conventional format may be used. Each well may optionally contain one or a mixture of ligands combined with the target in a suitable solution and the solution is then subjected to ESI-MS under conditions that maintain the structure of the target and the association of the target with the ligands. Mixtures of 2 to about 50 ligands, preferably 2 to about 20 ligands, more preferably 2-10 or 3-10 ligands may be combined with the target for each assay. Typically, the ligands and target will be combined with a buffer suitable to maintain the pH in a range such that a target biomolecule is biologically active.

The mass spectroscopy screening method of the invention generally utilizes ligand concentrations ranging from about 0.02 to about 10.0 mM and protein target concentrations of from about 0.5 micromolar to about 50 micromolar, preferably from about 1 to about 10 micromolar. The sample is generally prepared as a solution, preferably at biologically relevant conditions, including but not limited to, pH, solvent and salt concentration. The pH of the mass spectroscopy sample solution is preferably in a range such that the target biomolecule is biologically active. Typically this pH range is from about 5 to about 9, preferably from 6-8. Typically, samples do not contain additional organic modifiers that can stabilize the signal but destabilize weak noncovalent complexes. Under ESI conditions, high concentrations of inorganic salts or nonvolatile buffers which can mask the signal of the desired noncovalent complex of the target protein with a small molecule are preferably avoided. The needle voltage is generally in the range of about 2000-4000V, preferably from about 2500 to about 3000V. The orifice voltage (OR) should sufficiently low to allow the observation of noncovalent complexes and not so high as to break covalent bonds, and is typically in the range of about 40V to about 90V, preferably from about 55V tO about 70V.

If the target biomolecule contains a metal atom(s), for example certain enzymes, the mass spectrum may show the presence of the noncovalent complexes of the apo-species (e.g., metal free enzyme) with the target binding ligands instead of complexes with the holo-species (e.g., holo-enzyme). For such metal-containing biomolecules, the method of the invention is still capable of detecting 1:1 and higher order binding complexes and of identifying target binding ligands. However, for these biomolecules the dissociation constant may be altered by the absence of the metal.

Following the optional pre-screening step described above, one or more sets of target binding ligands are assembled. Candidate target binding ligands that have detectable binding, preferably by mass spectrometry, can be re-assayed using mass spectrometry for their ability to compete with each other for a common target binding site. Those molecules that compete with each other for a single site are defined as a target binding set. This classifies a set of binders as those binders that can compete with each other for only one of many target binding sites. The number of unique target binding sites that are present on the target, e.g. a target biomolecule, limits the number of such sets. Typically, a mixture of a target biomolecule and two or more target binding ligands are mixed in a suitable buffer and assayed by mass spectroscopy, preferably by ESI-MS, under conditions that maintain a non-covalent association of the ligands with the target in the mass spectrometer. For non-competitively binding ligands, the mass spectrum shows peaks for the target biomolecule, target associated with one ligand, target associated with a second ligand and target associated with both ligands (because the two ligands bind at different binding sites). For competitively binding ligands, the mass spectrum shows peaks for the target biomolecule, target associated with one ligand, target associated with a second ligand, but no peak for target associated with both ligands (because the two ligands bind at the same binding site). Pairwise assays allow the assembly of sets of target binding ligands where each member of the set competes with other members of the set for binding to a specific site on the target. Alternatively, three or more ligands can be assayed simultaneously by observing the corresponding peaks in a mass spectrum.

The candidate target binding ligands may be simple hydrogen bond donors, hydrogen bond acceptors, positively charged cations, negatively charged anions, zwitterions, and lipophilic compounds containing, but not limited to the following common functional groups, aldehydes, amines, amidines, guanidines, hydrazines, hydrazones, amides, cabamates, carbonates, esters, ureidos, sulfonamides, alcohols, carboxylic acids, thiols, aryl halides, alkanes, alkenes, alkynes, arenes, heteroarenes, heterocycles, ketones, ethers, thioethers and/or oximes.

Candidate target binding ligands are selected inter alia on the basis of size and molecular diversity. The ligands may have a diversity of shapes (e.g., flat aromatic rings(s), puckered aliphatic rings(s), straight and branched chain aliphatics with single, double, or triple bonds) and diverse functional groups (e.g., carboxylic acids, esters, ethers, amines, aldehydes, ketones, and various heterocyclic rings).

Individual members of sets of target binding ligands, identified during the assembling steps, can then be linked, e.g. coupled, fused or cross-linked, in a variety of combinations using one or more linker elements to provide a library of potential high affinity binding linked ligands, whose building blocks represent the target binding ligands having the highest affinity for the target identified as sets of target binding ligands. The library of potential linked ligands can then be screened a second time to identify those members that exhibit the lowest dissociation constant for binding to the target. Since each member of a set of target binding ligands can be seen as a building block for a linked ligand and the candidate target binding ligand building blocks are initially pre-screened to select for a much smaller set of the most favorable binding building blocks, the most productive building block and linker combinations can be identified without the laborious task of screening all possible combinations of all building blocks coupled together by a set of linkers. The process of identifying high affinity drug lead compounds is therefore, greatly expedited.

Molecules from each set of target binding ligands are then selected and linked covalently to generate new linked ligands with enhanced binding properties. This can be done with an eye toward the synthetic ease of introducing a linking moiety. Linked ligands are prepared by linking two binding ligands together with a linker. This can be achieved by connecting one end of a flexible linker (e.g. a polymethylene chain) to a radical, derived by the cleavage of a C—H, N—H, O—H, S—H or other bond involving a hydrogen, of a target binding ligand. A similar connection of a member of another set of target binding ligands will generate a stable linked molecule. A series of compounds of increasing linker length, e.g., from 0-20 methylene (or e.g., alkyl, alkyl ether) units allows for a flexible linkage maximizing the number of low energy conformations of the two linked ligands. This enables the linked ligands to survey the greatest amount of 3-dimensional conformational space and consequently, it maximizes the probability of identifying a linkage that can simultaneously bind each ligand to the target protein in the correct relative orientation. When the linker length is optimal, a 1:1 complex of target protein and linked ligand will be observed in the mass spectrum. If the linker is not optimal, and/or does not allow for the correct simultaneous binding of the linked ligands, nonspecific associations will be detected in the mass spectrum as a complex of protein plus two or more linked ligands (e.g., [ligand-1]-(CH₂)ₙ₋[ligand-2]:target:[ligand-1]-(CH₂)ₙ₋[ligand-2] and n is, for example, 1-20), where the linked ligands (i.e. ligand-1 and ligand-2) appear to act independently and in a similar fashion to an equimolar mixture of the two unlinked ligands.

Linking is preferably accomplished in a manner that maintains the relative spatial orientation of the ligands to one another and to the target molecule. Suitable linkers for use in this invention and synthetic methods of preparing them are described, for example, in WO9949314 or U.S. Pat. No. 6,335,155, which are incorporated herein by reference in their entirety. Linking includes forming direct single and multiple bonds between two ligands, as well as forming fused compounds, for example, by combining the two ligands into a single molecule having a molecular weight less than the sum of the two component ligands. Fusing two molecules eliminates any molecular redundancies that exist between them when forming a new fused molecule. As an example, methoxybenzene and benzene can be linked to form 2-methoxynaphthalene where two carbon atoms and four hydrogen atoms have become structurally redundant. Such linked molecules are preferred to maximize the functionality found in the molecules linked to form them.

Other suitable linking groups will be apparent to those having skill in this field. One or more molecules from a set of target binder ligands are chemically linked to one or more molecules from at least one other distinct set of target binder ligands to generate a new molecule with enhanced binding properties. The enhanced binding properties of the linked ligand can be recognized as an ability of the linked ligand to simultaneously compete with both of the target binding ligands from which it was formed. When the linked ligand and unlinked target binding ligands are studied at the same concentration, enhanced binding properties are recognized as greater signal intensity at the mass of the noncovalent complex of the target biomolecule and the linked ligand in comparison to either of the masses of the unlinked target binding ligand-target biomolecule complexes. An additional sign of enhanced binding is a decrease in the equilibrium constant for the dissociation of the noncovalent complex between a linked ligand and a target biomolecule over that of each unlinked target binding ligand-target biomolecule complex. The equilibrium binding constant can be calculated for each charge state from the relative peak intensities of each species in the equilibrium. The presence of a 2:1 complex of linked ligand and target biomolecule is an indication that the linkage is not optimal and each component in the linked ligand is binding independently of the other. Preferably, the relative proximity of the binding sites for two or more sets of binders is within thirty (30) Angstroms to link the binders from each site to improve the likelihood of identifying a drug lead simultaneously encompassing these two binding sites on the target biomolecule.

In one preferred embodiment, linkers allow the formation of a linked ligand library that can map the three-dimensional requirements for binding to a target binding site. Further, the linker preferably allows one to systematically vary the spatial presentation of the linked ligands while requiring minimal changes in the linker structure. The linker will also preferably allow coverage of the target binding site accessible to the linked ligand, sample discrete but overlapping regions of the target binding site and use minimal linker diversity so that binding differences are more readily attributable to structural variations. For example, a phenylacetylene core having variable length alkylene chains can be prepared according to the scheme shown below to prepare linkers having two arms oriented in the ortho, meta or para positions and bearing functional groups, for example, guanidino and carboxylic acid functional groups, for bonding to the two target binding ligands at each end of the two arms. In this scheme, n and m are independent integers from 1 to 4. However, in other embodiments, n and m may range up to about 10.

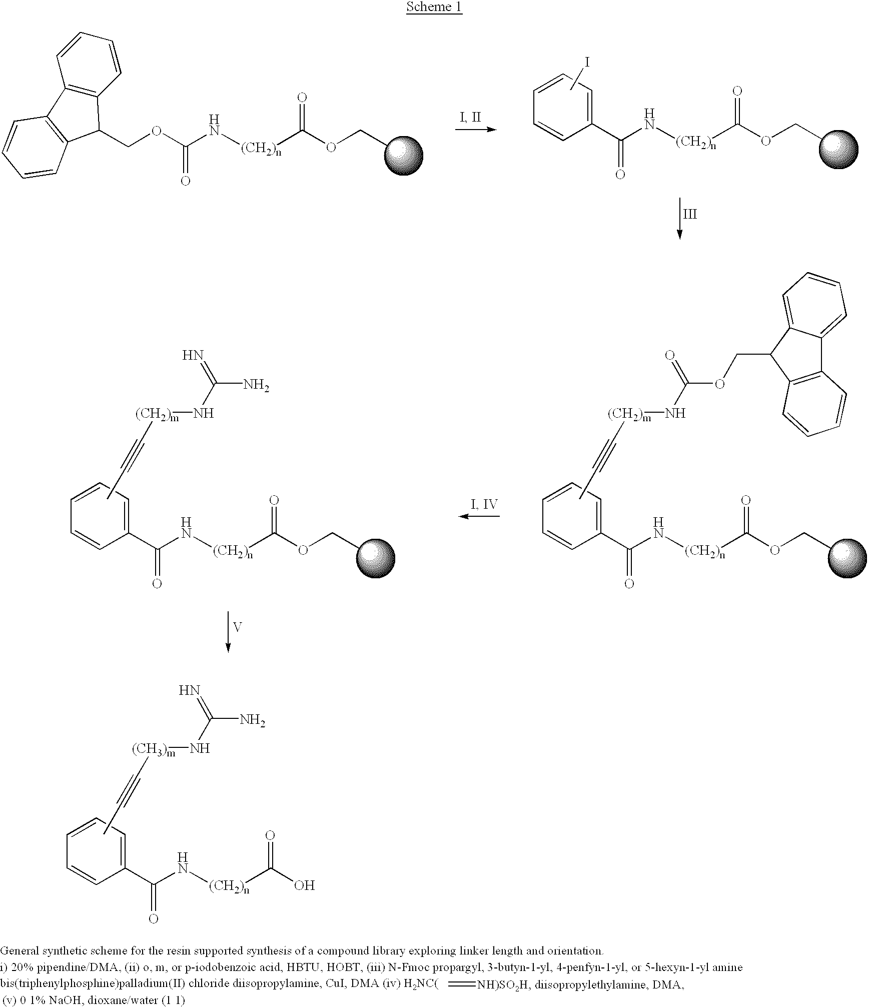

A member of a set of target binding ligands may also be covalently linked, e.g. fused, to another member of the same set of binders. Since both ligands in this embodiment bind to the same site, this type of ligand may be expected to enhance the binding properties of a new linked ligand by further optimizing the binding affinity at that site. This new ligand may then be further linked to a member from a different set of target binding ligands as described above to form a linked ligand. Linking two members of a set is particularly preferred when the two members have different functional groups with capacity for hydrogen bond donation, hydrogen bond acceptance, forming a cation at physiological pH, forming an anionic species at physiological pH, or hydrophobic interactions.

Linked ligands have enhanced binding properties. These enhanced binding properties can be recognized as an ability of the linked ligand to simultaneously compete for binding with both unlinked target binding ligands on a single target biomolecule. When the linked ligand and unlinked target binding ligands are studied at the same concentration, enhanced binding properties are recognized as greater signal intensity at the mass of the noncovalent complex of the target biomolecule and the linked ligand in comparison to the masses of either of the unlinked target binding ligand-target biomolecule complexes. An additional sign of enhanced binding is an decrease in the disassociation constant of the noncovalent complex between a linked ligand and a target biomolecule over that of each unlinked target binding ligand-target biomolecule complex. The disassociation constant can be calculated for each charge state from the relative peak intensities of each species in the equilibrium. The presence of a 2:1 complex of linked ligand and target biomolecule is an indication that the linkage is not optimal and each ligand in the linked ligand can bind independently of the other.

Preferably, each of the candidate target binding ligands, and therefore each of the target binding ligands assembled therefrom, has a molecular weight such that any subsequent combination into a linked ligand will provide a linked ligand that preferably has a molecular weight less than 2,000 daltons, more preferably less than 1,000 daltons, even more preferably in the range of 200-700 daltons. Consequently, the members of the library or libraries of candidate target binding ligands preferably have a molecular weight less than 600 daltons, more preferably less than 300 daltons, even more preferably in the range of 50-200 daltons.

Preferably, the candidate linked ligands have a K_{d} for the target molecule of less than about 500 nM, preferably less than 200 nM, more preferably less than 100 nM, still more preferably less than 50 nM.

More preferred are candidate target binding ligands having a molecular weight of less than 600 daltons and which are selected for their ability to mimic naturally occurring amino acid side-chains, including any modifications to those side-chains that are found in post-translational modifications of protein biomolecules. These candidate target binding ligands are likely to form non-covalent complexes with a target biomolecule, since protein molecules are known to form non-covalent complexes with target biomolecules including other protein(s), DNA and RNA via a series of interactions which put amino acid side-chains in contact with the target biomolecule.

Target biological molecules that find use in the described methods include, for example, proteins, nucleic acids, and saccharides—preferably proteins. Preferred target biomolecules include human or human pathogen proteins, especially enzymes, human hormones, human receptors, and fragments thereof.

Suitable target biomolecules are available (either commercially, recombinantly, synthetically or otherwise) in sufficient quantities for use in binding assays and for which there is some interest for identifying a high affinity binding partner. Target biomolecules include proteins, such as human proteins or human pathogen proteins that may be associated with a human disease condition, such as cell surface and soluble receptor proteins, such as cell surface receptors, enzymes, such as proteases, matrix metalloproteinases such as stromelysins, gelatinases and collagenases, clotting factors, serine/threonine kinases and dephosphorylases, tyrosine kinases and dephosphorylases, bacterial enzymes, fungal enzymes and viral enzymes, as well as signal transduction molecules, transcription factors, proteins associated with DNA and/or RNA synthesis or degradation, immunoglobulins, hormones, receptors for cytokines including, for example, erythropoietin/EPO, granulocyte colony stimulating receptor, granulocyte macrophage colony stimulating receptor, thrombopoietin (TPO), IL-1, IL-2, IL-3, IL-4, IL-5, IL-6, IL-10, IL-11, IL-12, growth hormone, prolactin, human placental lactogen (LPL), CNTF, octostatin, chemokines and their receptors such as RANTES, MIPI-(x, IL-8, ligands and receptors for tyrosine kinases such as insulin, insulin-like growth factor I (IGF-1), epidermal growth factor (EGF), heregulin-a and heregulin-b, vascular endothelial growth factors (VEGF) 1, 2, and 3, placental growth factor (PLGF), tissue growth factors (TGF-(X and TGF-P), other hormones and receptors such as bone morphogenic factors, folical stimulating hormone (FSH), and leutinizing hormone (LH), tissue necrosis factor (TNF), apoptosis factors (AP-1 and AP-2), nucleic acids, including both DNA and RNA, saccharide complexes, etc.

Further, additional sets of target binding ligands can be formed or assembled and linked to the sets of target binding ligands described above. For example, two sets of ligands (a first set and a second set) that bind to different target binding sites can be assembled as described above. Two further sets (a third set and a fourth set) can then be assembled where each member of the third and fourth sets competes with all members of the first set of ligands, but the members of the third set do not compete with members of the fourth set (and vice versa, all members of the fourth set also compete with members of the first set but do not compete with members of the third set) for the same target binding site. The sets can be readily determined by the mass spectroscopy assays described above. In general, the additional sets will be ligands that are sterically or physically smaller or that contain fewer functional groups than the ligands of the first set to allow ligands from the third set and the fourth set to bind within the same binding site simultaneously. Linked ligands formed by covalently linking or fusing at least one member of a third (or fourth) set with at least one member of a second set can be assayed for binding affinity as described herein.

To facilitate processing of data, computer programs may be used to transfer and automatically process the data sets. The analysis of the data can be facilitated by formatting the data so that the individual spectra are rapidly viewed and compared to the spectrum of the control sample containing only the vehicle for the added compound, but no added compound.

Deconstructive Embodiment

According to one embodiment of the invention (a deconstruction embodiment), at least a portion of the candidate target binding ligands are identified by considering a parent molecule, e.g. an existing drug or drug lead molecule, as a collection of stable fragment molecules and testing the binding of the stable fragment molecules to a target biomolecule (e.g., protein, nucleic acid, etc.) by determining, with mass spectrometry, the ability of each stable fragment molecule to compete for binding with the parent molecule, the stoichiometry of the binding of the parent molecule and stable fragment molecule, and the binding affinities of the parent and stable fragment molecule with the target molecule. Competition of the stable fragment molecules (alone or in combination) with the parent molecule is studied to determine which fragments play a role in the binding of the parent molecule to the target.

Competitive binding of a target biomolecule with libraries of candidate target binding ligands that are not known to bind the target in competition with stable fragment molecules can be carried out in order to identify set(s) of target binding ligands. Competition of candidate target binding ligands with stable fragment molecules of a parent molecule is used to identify those candidate target binding ligands that compete with a parent or fragment thereof for binding to the target. This enables the construction of novel drugs or lead molecules where candidate target binding ligands have been substituted for stable fragment molecules derived from a parent compound. The method also enables one to study the competitive association of target(s) with libraries of molecules not known to associate with the target.

Any existing parent molecule (drug lead or drug molecule) can be envisioned as one or more combinations of substructural units that contribute a portion of the binding energy of the parent molecule. The formation of these substructural units generally involves mentally breaking one or more chemical bonds to generate a substructural unit that would be unstable because of unfilled valencies (e.g. radicals). The valencies of these substructural units are then mentally filled by the addition of a functional group or groups to generate one or more compounds (or libraries of compounds) of stable fragment molecules to be evaluated as target binding ligands. The simplest choice of functional group to satisfy the valency of the substructural unit(s) would be a hydrogen atom. Other simple functional groups or radicals can be chosen to reflect the portion of the chemical bond that was broken to form the substructural unit so as to most closely mimic the functionality and binding properties of the parent molecule as the sum of the properties of the stable fragment molecules that are generated. Individual stable fragment molecules within these libraries are generally purchased or can be synthesized using well known chemical methods. This conceptual process of identifying stable fragment molecules for consideration as target binding ligands is analogous to the well known process of retrosynthetic analysis and the concept of synthons as taught to modem synthetic chemists See, for example, Corey (1967).

As an example, 2-methoxynaphthylene can be envisioned as a combination of 1,4-butadiene and methoxybenzene or as a combination of benzene and methoxybenzene. Because each combination of the stable fragment molecules can be recombined to form the parent molecule and the binding properties of the parent molecule are the sum of the properties of its stable fragment molecules, consideration should be given in choosing stable fragment molecules to have their combined properties closely reflect the properties of the parent molecule.

While not being bound by any particular theory, it is believed that a parent molecule can be viewed as a combination of substructural units such that the properties of the whole are the sum of the properties of the substructural units. This is true at the structural level in terms of molecular connectivity and three-dimensional presentation of a pharmacophore (i.e., the substructural unit(s) of the parent molecule which is (are) in contact with the target biomolecule and contribute(s) to the binding energy). The affinity of the parent molecule for a target biomolecule at the protein, DNA, RNA level is related to the energetics of the equilibrium binding interaction between the parent (e.g. drug) and target biomolecule (i.e. target) can be expressed by the following equation:

K=[Drug:Target]/([Drug]×[Target]).

In this equation, K is the equilibrium constant for the association of drug with target, [Drug:Target] is the concentration of the complex of drug and target. [Drug] is the concentration of free drug and [Target] is the concentration of free target at equilibrium. In the method of the invention, equilibrium is established in solution and mass spectroscopy is used to rapidly convert the equilibrium mixture from solution to ions in the gas phase without perturbing the equilibrium. In the mass spectrometer, the ratio of the [Drug:Target]/[Target] is observed as the ratio of the intensity of the ions observed for the Drug:Target complex and the Target in the mass spectrum. The concentration of free drug can be calculated as the difference between the total concentration of [Drug] added to the test solution and the relative concentration of the [Drug:Target] complex. The same equilibrium expression holds true for the association or binding of a parent molecule, candidate target binding ligand or stable fragment molecule in equilibrium with a target biomolecule.

The free energy of this association, ΔG, is related to this equilibrium constant by ΔG=−RT ln K and this free energy can be seen as the sum of the free energies for the interactions of the substructural units plus an additional term which accounts for the entropic and energetic effects of linking the stable fragment molecules into the drug molecule. This latter term also accounts for molecular redundancies or overlaps which occur in recombining the stable fragment molecules into the drug molecule.

ΔG_{Drug}=ΣΔG_{fragments}+ΣΔG_{linking}

Consequently, an equilibrium expression can be derived from the dissociation of the fragment molecule-target biomolecule complex.

K_{fragment}=([Fragment]×[Target])/ [Fragment:Target]

In this equation, [Fragment:Target] is the concentration of the complex of fragment molecule and target biomolecule, [Fragment] is the concentration of free fragment molecule, and [Target] is the concentration of free target biomolecule at equilibrium.

As a further example, the drug, indomethacin can be fragmented into several simpler more synthetically or commercially accessible molecules (see below) that may have existed before indomethacin. The association of the parent molecule (e.g., indomethacin) with its protein target (prostaglandin synthetase) can be studied, as well as the association of any of the combinations of stable fragment molecules alone (forming a binary complex with the target biomolecule), in common (forming ternary or higher order complexes of fragment molecules with the target biomolecule), or in competition (forming a series of binary and/or higher order complexes of molecules with target biomolecule).

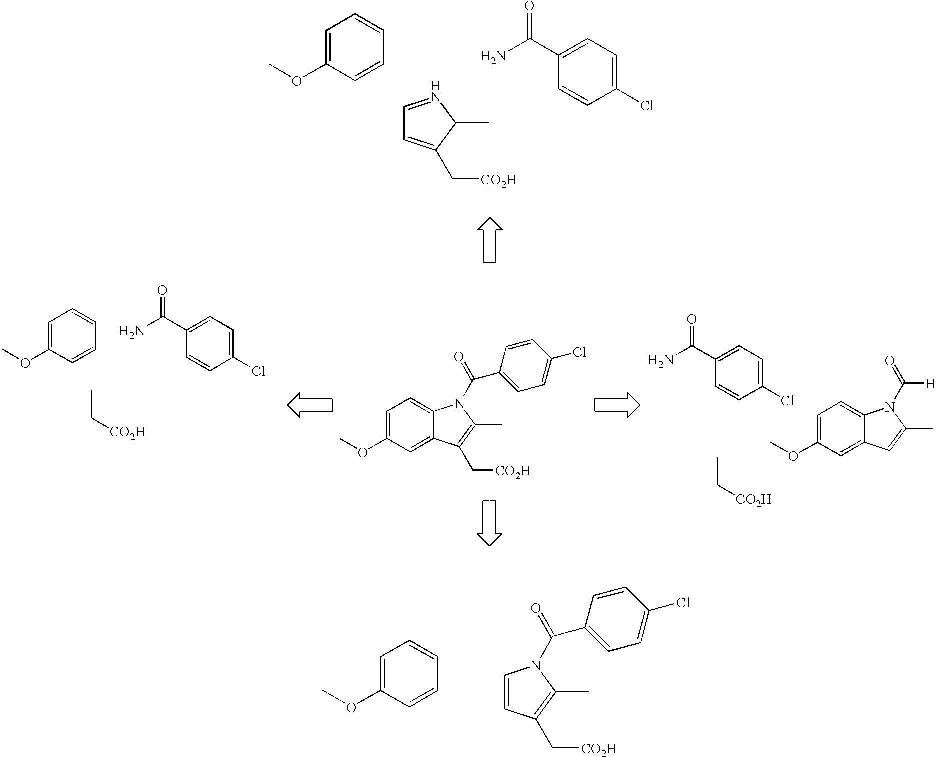

Similarly, a protein can be seen as a collection of amino acids and can be fragmented into a sets of fragment molecules formed by breaking the amino acid sidechain from the protein amide backbone at the Cα-Cβ bond. The resulting fragment molecules can be grouped into sets based on their molecular properties. For example, one set may contain uncharged hydrogen bond donors, a second set may contain uncharged hydrogen bond acceptors, a third set may contain molecules which would bear a substantial degree of positive charge at physiological pH, a fourth set may contain molecules which would bear a substantial degree of negative charge at physiological pH and a fifth set may contain fragment molecules which are hydrophobic or lipophylic. In this example, members of the first set are fragment molecules derived from serine, threonine, asparagine, glutamine, tyrosine and tryptophan, members of the second set are fragment molecules derived from asparagine, glutamine, and possibly serine, threonine, tyrosine and tryptophan, members of the third set are positively charged fragment molecules derived from histidine, lysine, arginine, members of the fourth set are fragment molecules derived from negatively charged fragment molecules derived from aspartic acid, glutamic acid, and members of the fifth set are fragment molecules derived from alanine, valine, leucine, isoleucine, methionine, phenylalanine and possibly tryptophan and tyrosine. Each of these sets of molecules forms the basis of a library of compounds which are chosen to reflect or mimic the function of protein fragment molecules and amino acid sidechains. These libraries are the basis of candidate target binding ligands for a portion of the parent molecule. Consequently, a series of five or more libraries can include compounds capable of forming noncovalent complexes with target biomolecules as uncharged hydrogen bond donors, uncharged hydrogen bond acceptors, cations that bear a substantial degree of positive charge at physiological pH; an ions that bear a substantial degree of negative charge at physiological pH; and hydrophobic or lipophylic groups. The tables below show these sidechain structures as well as surrogate structures therefor that are the basis of corresponding libraries of compounds which reflect or mimic the function of these sidechains. The tables also show examples of specific compounds that might form such a library. Obviously, other specific compounds that reflect or mimic the function of these sidechains are possible and can be used in the libraries. For certain biomolecules that are known or suspected to have a particular function or have homology to a biomolecule known to have a particular function (e.g. an enzyme) an additional library can be constructed from compounds whose functional groups and overall properties are predicted interact with the functional portion of the target biomolecule.

Table 1 contains a description of a library of positively charged compounds that are cations and as such are capable of mimicking the positively charged sidechains of the amino acids lysine, arginine and histidine in binding to a target biomolecule.

Table 2 contains a description of a library of hydrophobic or lipophylic compounds which are capable of mimicking the hydrophobic or lipophylic nature of the sidechains of the amino acids alanine, valine, leucine, isoleucine, methionine, proline, phenyl alanine, tyrosine and tryptophan in binding to a target biomolecule.

Table 3 contains a description of a library of negatively charged compounds which are anions and as such are capable of mimicking the negatively charged sidechains of the amino acids aspartic acid, glutamic acid, and the naturally occurring sulfate and/or phosphate esters of serine, threonine and tyrosine in binding to a target biomolecule.

Table 4 contains a description of a library of uncharged compounds which are hydrogen bond acceptors and as such are capable of mimicking the hydrogen bonding capacity of the sidechains of the amino acids serine, threonine, tyro sine, tryptophan, asparagine and glutamine in binding to a target biomolecule.

Table 5 contains a description of a library of uncharged compounds which are hydrogen bond donors and as such are capable of mimicking the hydrogen bonding capacity of the sidechains of the amino acids serine, threonine, tyrosine, tryptophan, asparagine and glutamine in binding to a target biomolecule.

The preparation of compounds as potential target binding ligands and for inclusion in the libraries described above is accomplished using well known organic synthetic methods, such as those described, for example, in Advanced Organic Chemistry : Reactions, Mechanisms, and Structure. 4th ed., ed.:March, Jerry New York: Wiley,c1992 and Comprehensive Organic Chemistry: The Synthesis and Reactions of Organic Compounds. 1st ed., ed: Barton, Derek Harold Richard, New York: Pergamon Press, 1979.

TABLE 1
Positively Charged Amino Acids and Surrogates

| | |
|---|---|
| 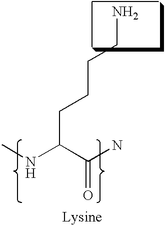 | 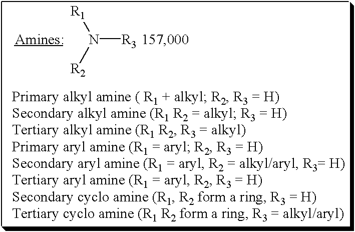 |
| 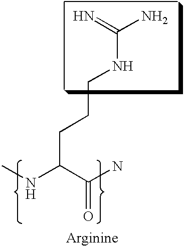 | 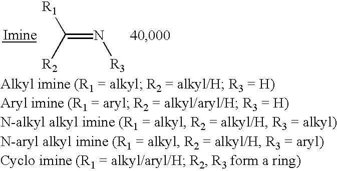 |
| 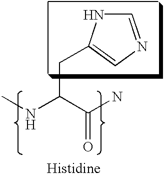 | 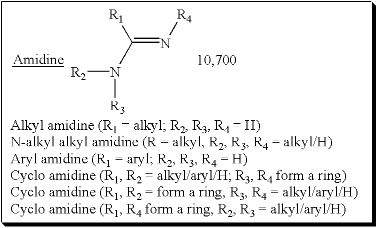 |
| | 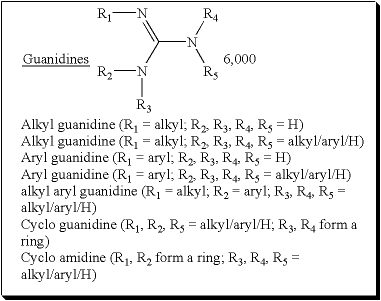 |

Other functional groups: Hydrazines, hydrazones, hydroxyl amines.

TABLE 2

| Lipophylic Sidechain Residues | Possible surrogate functional groups | | |
|---|---|---|---|
| 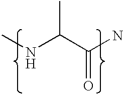 | 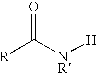 | 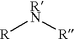 | 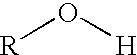 |
| Alanine | Carboxamide, Lactam | Thiocarboxamide | Alcohol |
| 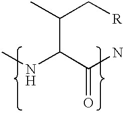 | -Alkyl -Linear -Branched -Cycloalkyl -Aryl -Arylalkyl -Linear -Branched Carboximide -Linear | -Linear -Branched -Cycloalkyl -Aryl -Arylalkyl -Linear -Branched Thiourea -Linear -Branched | |
| # -Linear -Branched -Cycloalkyl -Aryl -Arylalkyl -Linear -Branched Phenol, substituted -Linear | | | |
| Valine, R = H and iso-Leucine, R = CH₃ | -Branched | -Cycloalkyl | -Branched |
| 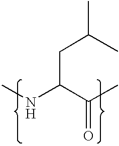 | -Cycloalkyl -Aryl -Arylalkyl -Linear -Branched Urea -Alkyl -Linear -Branched -Cycloalkyl | -Aryl -Arylalkyl -Linear -Branched Thiocarbamate -Linear -Branched -Cycloalkyl -Aryl -Arylalkyl -Linear | |
| # -Cycloalkyl -Aryl (fused) -Arylalkyl -Linear -Branched | | | |
| Leucine | -Aryl | -Branched | |
| 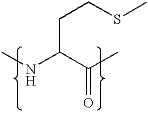 | -Alryalkyl -Linear -Branched Including cyclic examples where R and R′ are joined to form a mono or bicyclic heterocycle | Thiocarboximide -Linear -Branched -Cycloalkyl -Aryl -Arylalkyl -Linear -Branched Sulfonamide, Phosphoramide, Phosphonamide | |
| Methionine | | -Linear | |
| 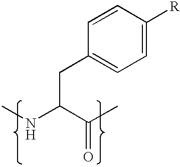 | 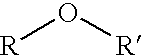 Ether -Linear -Branched -Cycloalkyl -Aryl -Arylalkyl -Linear -Branched Ester | -Branched -Cycloalkyl -Aryl -Arylalkyl -Linear -Branched Including cyclic examples where R and R′ are joined to form a mono or bicyclic heterocycle | |
| Phenylalanine, R = H and Tyrosine, R= OH | -Linear | | |
| 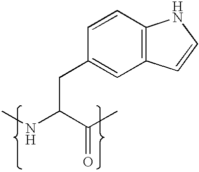 | -Branched -Cycloalkyl -Aryl -Arylalkyl -Linear -Branched Including cyclic examples where R and R′ are joined to form a mono or bicyclic heterocycle | | |
| Tryprophane | | | |

TABLE 3

| Negatively Charged Sidechains | Possible surrogate functional groups |
|---|---|
| 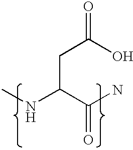 | Carboxylic acid -aliphatic -aromatic -heterocyclic N-Hydroxamic acid -aliphatic -aromatic -heterocyclic |
| Aspartic Acidsidechain carboxylic acid | |
| 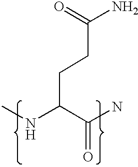 | Sulfuric acid monoester -aliphatic -aromatic -heterocyclic Sulfonic acid -aliphatic -aromatic -heterocyclic Sulfinic acid -aliphatic |
| Glutamic Acid sidechaincarboxylic acid | -aromatic |
| 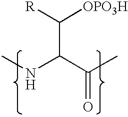 | -heterocyclic Phosphoric acid monoester -aliphatic -aromatic -heterocyclic Phosphonic acid monoester -aliphatic |
| PhosphoSerine, R = H and PhosphoThreonine, R = CH₃ | -aromatic |
| 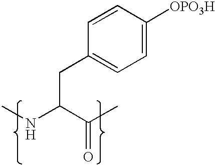 | -heterocyclic Phosphonic acid -aliphatic -aromatic -heterocyclic Tetrazoles -aliphatic -aromatic -heterocyclic |
| PhosphoTyrosine sidechain | |

TABLE 4

| Uncharged Hydrogen Bond Acceptors | Possible surrogate functional groups | | |
|---|---|---|---|
| 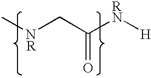 | 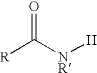 | 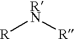 | 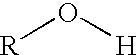 |
| Mainchain carboxamide, R = H, CH₂ | Carboxamide, Lactam | Thiocarboxamide | Alcohol |
| 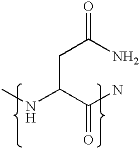 | Urea Carbamate, Urethane N-Hydroxy urethane Carboximide 2,4-Oxazolidinedione 2,4-Thiazolidinedione Hydantoin Pyrimidine, Uracil N-Acylurea Semicarbazide Barbituric acid | | |
| # Thiourea Thiocarbamate Thiocarboximide Sulfonamide Phosphoramide Phosphonamide Benzamidazole Indole Pyrole Imidazole Including cyclic examples | Phenol N-Hydroxyamic acid N-Hydroxy carboximide N-Hydroxyurea N-Hydroxyurethane | | |
| Asparigine sidechain carboxamide | N-Acylhydrazine | where R and R′ are joined to | |
| 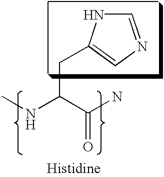 | N-Acylhydrazone N-Sulfonamide N-Sulfonylurea N-Hydroxamic acid N-Hydroxamic acid ester Including cyclic examples where R and R′ are joined to form a mono or bicyclic heterocycle | form a mono or bicyclic heterocycle | |
| Glutamine sidechain carboxamide | | | |
| 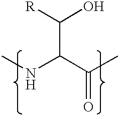 | | | |
| Serine, R= H and Threonine, R= CH₃ | | | |
| 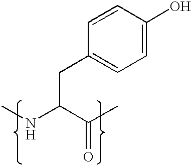 | | | |
| Tyrosine sidechain Hydroxyl | | | |

TABLE 5

| Uncharged Hydrogen Bond Donors | Possible surrogate functional groups | | |
|---|---|---|---|
| 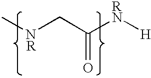 | 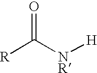 | 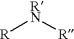 | 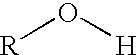 |
| Mainchain carboxamide, R = H, CH₂ | Carboxamide, Lactam | Thiocarboxamide | Alcohol |
| 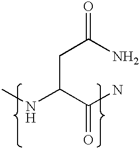 | Urea Carbamate, Urethane N-Hydroxy urethane Carboximide 2,4-Oxazolidinedione 2,4-Thiazolidinedione Hydantoin Thiohydantoin Pyrimidine, Uracil N-Acylurea Semicarbazide | | |
| # Thiourea Thiocarbamate Thiocarboximide Sulfonamide Phosphoramide Phosphonamide Benzamidazole Indole Pyrol Imidazole | Phenol N-Hydroxamic acid N-Hydroxy carboximide N-Hydroxyurea N-Hydroxyurethane | | |
| Asparigine sidechain carboxamide | Barbituric acid | Including cyclic examples | |
| 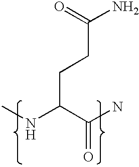 | N-Aclhydrazine N-Acylhydrazone N-Sulfonamide N-Sulfonylurea N-Hydroxamic acid N-Hydroxamic acid ester Including cyclic examples where R and R′ are joined to form a mono or bicyclic heterocycle | where R and R′ are joined to form a mono or bicyclic heterocycle | |
| Glutamine sidechaincarboxamide | | | |
| 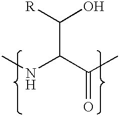 | | | |
| Serine, R= H and Threonine, R= CH₃ | | | |
| 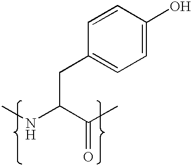 | | | |
| Tyrosine sidechain Hydroxyl | | | |
| 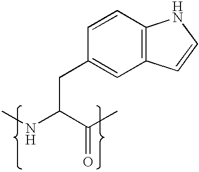 | | | |
| Tryprophane sidechain indole NH | | | |

After a parent molecule has been envisioned as several fragment molecules, the binding of corresponding ligands can be studied in competition with other candidate target binding ligands to determine the membership of each ligand in a set of target binding ligands. Linking the various members of sets of target binding ligands that into novel drugs or drug lead candidates can also be guided by molecular modeling and consideration of the structure of the parent molecule. Thus, membership of a ligand in a set of target binding ligands can be coupled with knowledge of the relative proximity and orientation of two or more fragment molecules gained from their connectivity within the parent molecule to guide the linking of individual members of the sets of target binding ligands.

Constructive Embodiment

In the case of novel targets with no known parent drug or drug lead molecules, the method of the invention permits rapid identification of molecules that bind to a target, even a novel target. The molecules can then be assayed with the target, e.g., in vivo, in vitro, ELISA or cell-based assay, to determine biological activity.

The method of the invention permits the rapid identification of molecules that bind to a target biomolecule when there is no parent molecule from which to design stable fragment molecules by the optional pre-screening process described above. Target binding ligands that have detectable binding can be re-assayed using mass spectrometry for their ability to compete with each other for a common target biomolecule binding site. Those candidate target binding ligands that compete with each other for a single site are defined as a set of target binding ligands as above. Two or more ligands from distinct sets or from the same set of target binding ligands are then be linked together to form new linked ligands. Linking is accomplished as described above.

Linked ligands that have been identified using the method of the invention may be subsequently tested using any known biological assay to identify function-blocking binders. The method of the invention, therefore, enables the rapid identification of molecules that bind to the target. These molecules can then be assayed for their ability to block biological function in biological assays (e.g., in vitro ELISA or cell based assays). Knowledge of the K_{d} measured by mass spectrometry can guide the optimum assay concentration needed to reduce false negatives. Each molecule that binds and has function can be used as a starting point for more traditional drug development techniques.

The binding of a second ligand can be measured in the presence of a first ligand that is already bound to the target. The ability to simultaneously identify binding sites of different fragment molecules allows 1) negative and positive cooperative binding between ligands to be defined and 2) new drugs by linking two or more ligands into a single compound while maintaining a proper orientation of the ligands to one another and to their binding sites to be designed.

There are numerous advantages to the method of the present invention. First, because the method of the present invention identifies ligands by directly measuring binding to the target, the problem of false positives is significantly reduced. Second, the problem of false negatives is significantly reduced because the method can identify compounds that specifically bind to the target with a wide range of dissociation constants. Other advantages of the present invention result from the variety and detailed data provided about each ligand.

The following Examples illustrate preferred embodiments of the present invention and are not limiting of the specification and claims in any way.

### EXAMPLES

### Example 1

Stromelysin is a member of zinc-dependant enzymes known as matrix metalloproteinases (MMP). Matrix metalloproteinases are important for connective tissue remodeling or breakdown. Increased levels of MMP activity have been implicated in a number of diseases such as rheumatoid arthritis, cancer, and corneal ulceration. This makes stromelysin an attractive target for small molecule inhibitors.

There have been a number of reports on inhibitors for stromelysin. Hajduk et al (1997); Olejinczak et al (1997). NMR and ¹⁵N labeled stromelysin in the presence of saturating amounts of acetohydroxamic acid has been used to identify ligands that bind in the S₁′ site of stromelysin. The ligands were optimized by modifying their structure and measuring the affect the modifications had on their dissociation constants by NMR. The three-dimensional structure stromelysin with 4-phenylpyridine was solved. Using this NMR method, the hydroxamic acid moiety and the optimized biphenyl ligand were linked together creating a molecule with higher affinity than the sum of the combined affinities of the individual ligands. The dissociation constants obtained for each of the ligands were in close agreement of the values obtained by assay or calorimetric titration.

Candidate target binding ligands used in the Examples 1-3 are shown below. FIGS. 7A-7E are ligands that are known to bind in the S₁′ site on stromelysin. Ligands 7F-7K are alternative candidate target binding ligands that were tested by the use of competition experiments for binding in the S₁′ binding site. Candidate target binding ligands 7L-7BB are amides that were used for the structure activity relationship study. The relative dissociation constants for each for each ligand were measured to optimize the amide. Competition experiments were also performed to assure that each ligand bound in the same binding site as 4-phenylpyridine.

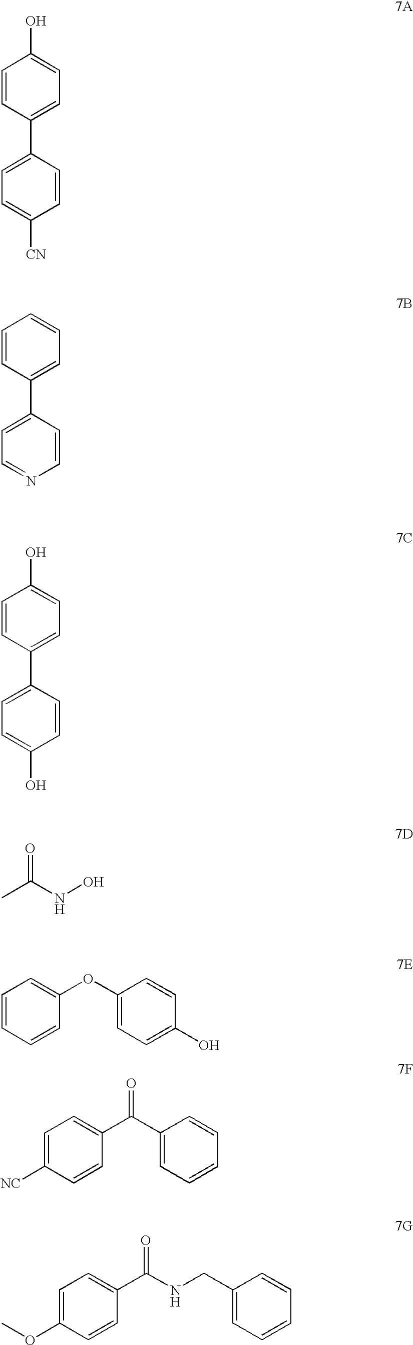

Acetohydroxamic acid was used as the ligand for the first binding site of stromelysin. It has been reported that complexation of zinc with acetohydroxamic acid prevents autocatalytic degradation of the protein. The mass of the acetohydroxamic acid is such that the mass of the binary complex of stromelysin and the acetohydroxamic acid does not appear in the same mass/charge range as the complexes of stromelysin and the ligand for the second binding site.

The ESI-MS measurements were recorded on a Perkin Elmer Sciex API III mass spectrometer. Samples were introduced via 75 μM i.d. fused silica capillary from a 50 μL syringe using a Harvard Apparatus Model 22-syringe pump at a flow rate of 1.500 μL/min. The orifice potential was set between 55 and 65V, and the interface heater was at 56° C. Mass spectra were obtained by averaging a sufficient number of scans to obtain adequate signal to noise. The concentrations of each of the initial set of ligands tested were concentrations equal to their reported K_{d} values.

A second ligand, 4-(4′-cyanophenyl)-phenol, was diluted with water from a 10 mM stock solution in DMSO and was equilibrated with stromelysin and acetohydroxamic acid for 1-2 hours at room temperature prior to analysis. The sample was analyzed by ESI-MS. For ions observed for the (M+17H)¹⁷⁺ were m/z of 1141, 1176, 1153 and 1159. The peak at 1141 corresponds to uncomplexed stromelysin. The m/z values of 1176 and 1153 correspond to stromelysin complexed with the acetohydroxamic acid and stromelysin complexed with 4-(4′-cyanophenyl)-phenol, respectively. The ternary complex of stromelysin with acetohydroxamic acid and the 4-(4′-cyanophenyl)-phenol ligand was observed at the m/z of 1159. There were no ions detected that corresponded to more than a 1:1 complex of either ligand with stromelysin indicating the interactions of the ligands with the protein were specific and not due an aggregation of ligands onto the protein as the solvent evaporated. Ions corresponding to these three complexes were also observed in other charge states of the spectrum.

The catalytic domain of stromelysin was prepared in accordance with well-known known procedures. The molecular mass was determined by electrospray ionization mass spectrometry to be 19,395.51. The stromelysin used for this study was in a solution of 20 mM Tris buffer, 5 mM CaCl₂, 0.02% NaN₃ at a pH of 7.5 and at a concentration of either 19.6 mg/mL or 21.7 mg/mL. Acetohydroxamic acid was used for the zinc-binding site.

The second step in the design process was the identification of a second ligand that binds to the target stromelysin at a site different from the binding site of acetohydroxamic acid. This may be accomplished by screening compounds for their ability to bind stromelysin in the presence of saturating amounts of acetohydroxamic acid.

The next step in the design process was to construct a ternary complex of the target stromelysin, the first ligand and the second ligand. This was accomplished by exposing the stromelysin target to the two ligands under conditions that result in complex formation.

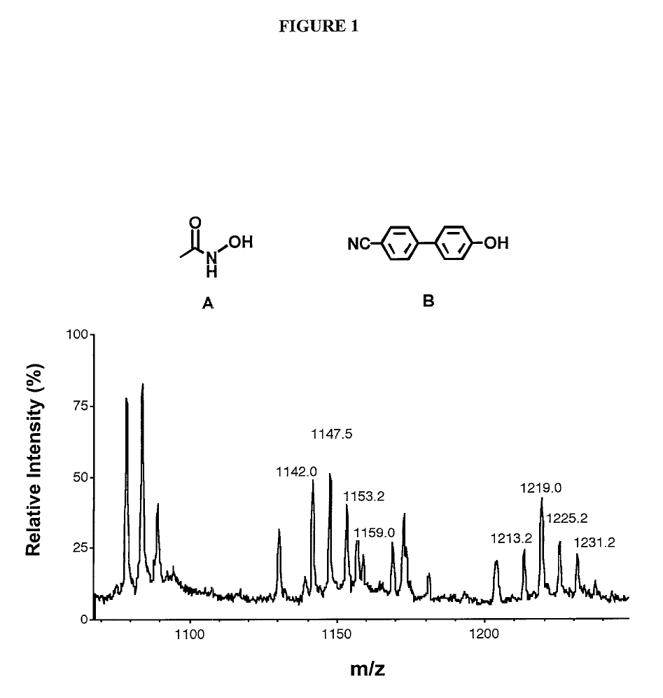

FIG. 1 shows a typical mass spectra recorded under conditions noncovalent complex forming conditions. Several different charged states are observed for noncovalent complexes between stromelysin, acetohydroxamic acid, 4-(4′-cyanophenyl)-phenol, and the ternary complex. The relative intensity of the different species present reflects their relative stabilities in solution. Ligands 7B, 7O, 7K and 7H were determined to be non-competitive binders; ligands 7G, 7Y, 7Z, 7BB, 7N, 7L and 7M were determined to be competitive binders forming a set of target binding ligands. Ligands 7P and 7S were non-binders. The binding constants for 7G (190 nM), 7Y (300 nM), 7Z (435 nM), 7BB (30 nM), 7N (370 nM), 7L (230) and 7M (310 nM) were determined.

Based on the known three-dimensional structure of ternary complexes with known drug lead molecules and the structure/activity relationships observed for the binding to stromelysin of known structural analogs of the second ligand, new molecules can be designed that link together, using linkers that are well known in the art—the acetohydroxamic acid to the second ligand.

Thus, the dissociation of a series of ligands were determined using ESI-MS. The dissociation of the ligands with known affinities for stromelysin were in close agreement with published values. The known ligands can be used to prepare novel ligands useful in identifying small molecule drug lead compounds.

### Example 2

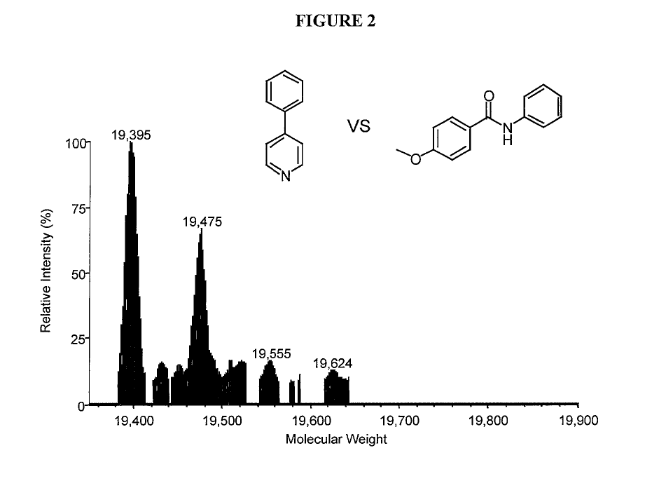

Example 2 was conducted in substantially the same manner as Example 1, using two different candidate target binding ligands. FIG. 2 shows a reconstructed mass spectrum of a competition experiment between 4-phenylpyridine and 4-methoxy-N-phenylbenzamide. Peaks corresponding to a noncovalent complex of each of these ligands complexed to the protein separately are observed. The mass corresponding to these ligands bound to the protein simultaneously is absent. These two ligands, therefore, compete for the same binding site on the protein. There is an absence of the masses corresponding to a greater than 1:1 complex of either ligand with stromelysin. This indicates the associations of each of the ligands are specific.

### Example 3

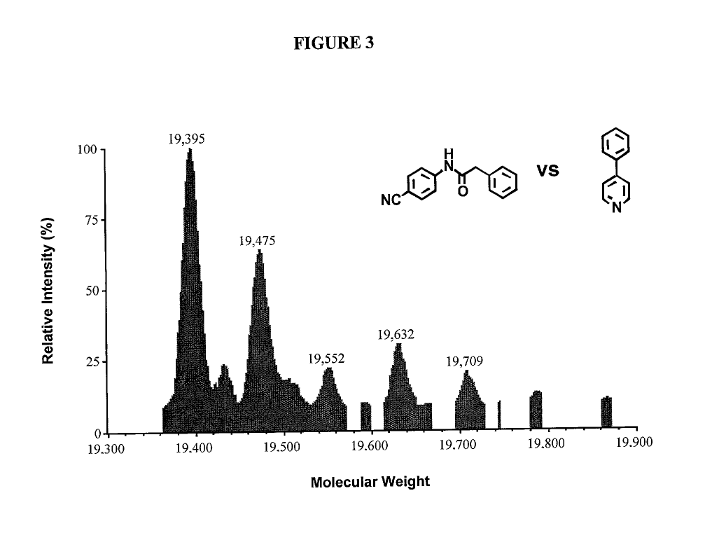

Example 3 was conducted in substantially the same manner as Example 1, using two different candidate target binding ligands. FIG. 3 shows a reconstructed mass spectra of the competition between 4-phenylpyridine and N-(4-cyanophenyl)-2-phenylacetamide. Masses corresponding to the ligands bound separately are present; however, a mass corresponding to the two ligands bound to the protein simultaneously is also present. This is a demonstration of two ligands that do not compete for the same binding site on the protein. Masses corresponding to a greater than 1:1 complex of either ligand with stromelysin is absent. This indicates that although the ligands do not bind on the same binding site, they bind to the protein specifically.

### Example 4

Linked ligands were then synthesized based on two sets of non-competitively binding ligands, i.e. acetohydroxamic acid and the ligands identified above to provide the linked ligands shown below. The tested binding affinity of these linked ligands ranged from 93 μM to 350 μM.

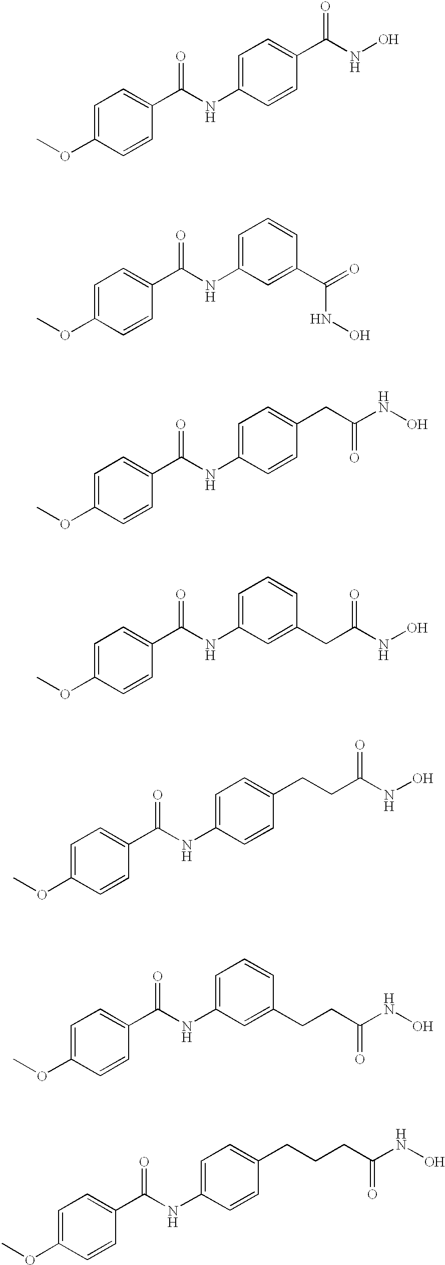

These linked ligands were synthesized generally according to the scheme shown below.

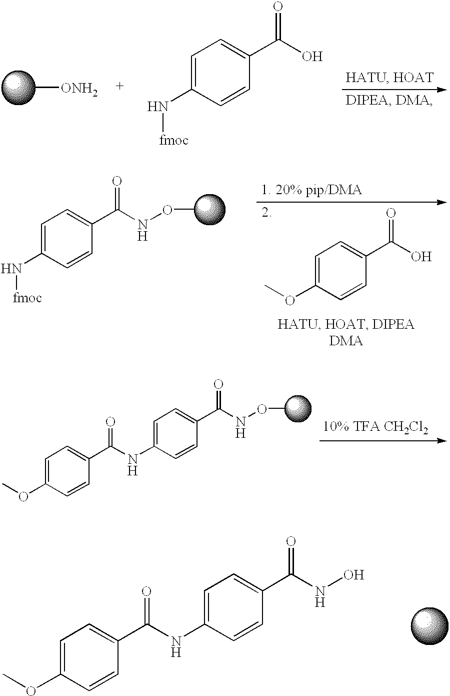

In this scheme, the “ball” represents a chlorotrityl group. See Chen, W., et al, (1997) Tetrahedron Letters, 38: 3311-3314.

### Example 5—PTP 1b

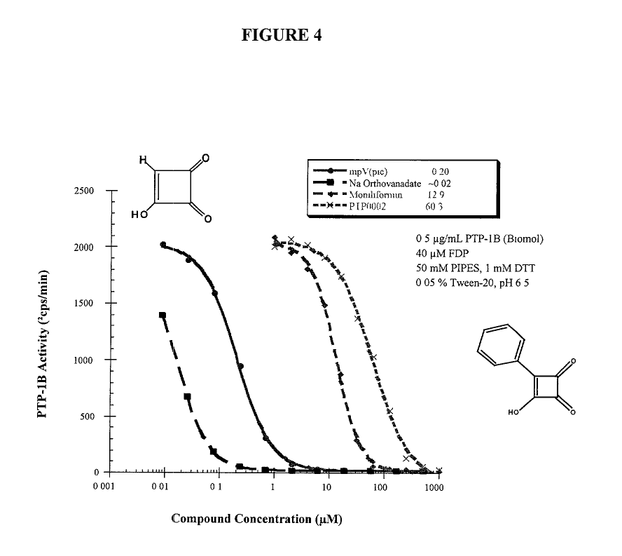

The candidate target binding ligands shown below were tested by mass spectrometry in a manner similar to Example 1 to determine whether any of the fragments bound specifically to the target biomolecule, protein tyrosine phosphatase 1b (PTP1b), observed 1:1 noncovalent complex. Fragment molecules were chosen based on either their ability to mimic the stabilized transition state of PTP1b with a tyrosine phosphate ester or based on their known or suspected ability to inhibit cysteine proteases and the homology or similarities in the active sites PTP1b and cysteine proteases. The transition state mimics included in the library are derivatives of moniliformin and deltic acid. Each of these compounds contain a planar arrangement of oxygen atoms around and electrophilic center, similar to the planar arrangement of oxygen atoms around an electrophilic phosphorous in the stabilized transition state complex of phosphotyrosine and PTP1b. Also contained in the library were various examples of some fragment molecules containing functional groups known or suspected to interact with an active site cysteine of cysteine proteases. Included in this example are aldehydes, α-ketoacids, boronic acids, and nitrites. Other fragment molecules could contain functional groups such as α-ketoesters, sulfonyl fluorides, haloketones, azabenzenes, and disulfides and other fragment molecules that contain functional groups known or suspected to react with mercaptans. It was determined by mass spectrometry that compounds 8A, 8D, and 8N all formed specific complexes with PTP1b. The binding constant (K_{d}) of 8A was determined to be 25 μM. The ability of these fragments to block the function of PTP1b was determined by a chromagenic substrate assay. Inhibition concentration curves are shown in FIG. 4.

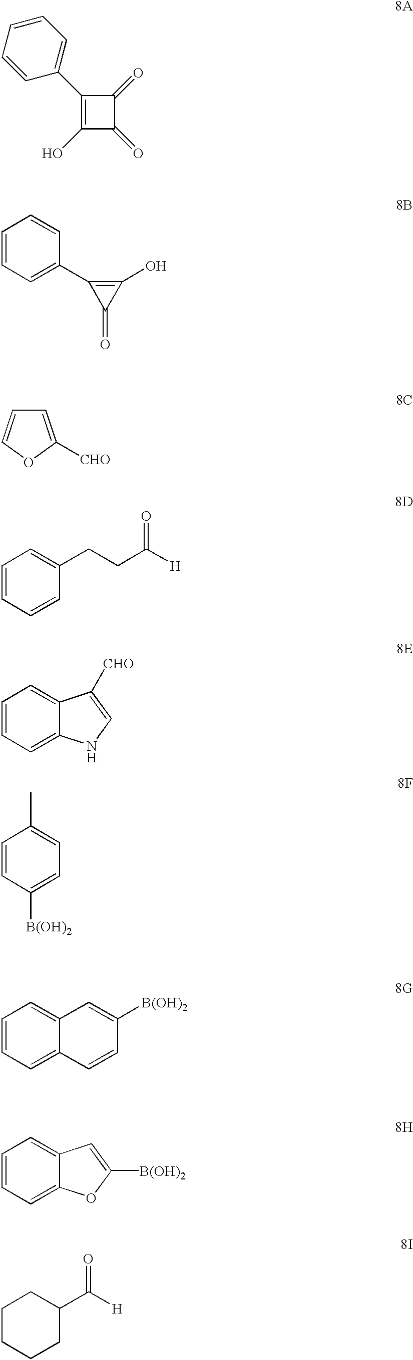

The present invention has been described with reference to preferred embodiments. Those embodiments are not limiting of the claims and specification in any way. One of ordinary skill in the art can readily envision changes, modifications and alterations to those embodiments that do not depart from the scope and spirit of the present invention.

### REFERENCES

1. Brown, Molecular Diversity, 2:217-222 (1996).

2. Cheng, X., Chen, R., Bruce, J. E., Schwartz, B. L., Anderson, G. A., Hofstadler, S. A., Gale, D. C., Smith, R. D., Using electrospray ionization FTICR mass spectrometry to study competitive binding of inhibitors to carbonic anhydrase, J. Am. Chem. Soc., 117:8859-60 (1995).

3. Gallop, et al., J. Med. Chem., 37:1233-51 (1994).

4. Ganem, B., Li, Y.-T., Henion, J. D., Detection of noncovalent receptor-ligand complexes by mass spectrometry, Am. Chem. Soc., 113:6294-6 (1991).

5. Gao, J., Cheng, X., Chen, R., Sigal, G. B., Bruce, J. E., Schwartz, B. L., Hofstadler, S. A., Anderson, G. A., Smith, R. D., Whitesides, G. M., Screening derivatized libraries for tight binding inhibitors to carbonic anhydrase II by electrospray ionization-mass spectrometry, J. Med. Chem., 39:1949-55 (1996).

6. Hajduk, P. J., Sheppard, G., Nettesheim, D. G., Olejniczak, E. T., Shuker, S. B., Meadows, R. P., Steinman, D. H., Carrera, G. M., Marcotte, P. A., Severin, J., Walter, K., Smith, H., Gubbins, E., Simmer, R., Holzman, T. F., Morgan, D. W., Davidsen, S. K., Summers, J. B., Fesik, S. W., Discovery of potent non-peptide inhibitors of stromelysin using SAR by NMR, J. Am. Chem., 119:5818-27(1997).

7. Hajduk, et al., J. Am. Chem. Soc., 119:5828-32 (1997).

8. Hu, P. F., Ye, Q.-Z., Loo, J. A., Calcium stoichoimetry determination for calcium binding proteins by electrospray ionization mass spectrometry, Anal. Chem., 66:4190-4 (1994).

9. Jorgensen, T. J. D., Roepstorff, P., Direct determination of solution binding constants for noncovalent complexes between bacterial cell wall peptide analogues and vacomycin group antibiotics by electrospray ionization mass spectrometry, Anal. Chem., 70:4427-32 (1998).

10. Loo, J. A., Studying noncovalent protein complexes by electrospray ionization mass spectrometry, Mass Spectrometry Reviews, 16:1-23 (1997).

11. Marcy, A. I., Biochemistry, 30:6476-83 (1991).

12. Matayoshi, E., et al., Science, 247:954-8 (1990).

13 Olejinczak, E. T., Hajduk, P. J., Marcotte, P. A., Nettesheim, D. G., Meadows, R. P., Edalji, R., Holzman, T. F., Fesik, S. W., Stromelysin inhibitors designed from weakly bound fragments: effects of linking and cooperativity, J. Am. Chem. Soc., 119:5828-32 (1997).

14. Shuker et al., Science, 274:1531-1534 (1996).

15. Corey, E. J., Pure Appl. Chem., 14:9 (1967).

What is claimed is:

1. A method for identifying compounds that bind to a target of interest, comprising:
(a) assembling a first set of target binding ligands that compete for non-covalent binding to a first binding site on the target;
(b) assembling a second set of target binding ligands that compete for non-covalent binding to a second binding site on the target;
(c) chemically linking at least one member of the first set and at least one member of the second set to provide a first set of linked ligands; and
(d) screening the set of linked ligands to identify members thereof that bind to the target.

2. The method of claim 1, wherein assembling step (a) or assembling step (b) comprises measuring non-covalent binding of target binding ligands to the target by mass spectroscopy.

3. The method of claim 2, wherein target binding ligands having a disassociation constant, K_{d}, equal to 500 μM or less are assembled into a set.

4. The method of claim 2 or 3, wherein the identified linked ligands have a disassociation constant, K_{d}, equal to 500 μM or less.

5. The method of any of the previous claims, wherein the first binding site is the same as the second binding site.

6. The method of any of the previous claims, wherein the first binding site is not the same as the second binding site.

7. The method of any of the previous claims, wherein assembling step (b) comprises determining binding of target binding ligands to the target having at least one member of the first set of target binding ligands bound thereto.

8. The method of any of the previous claims, wherein the target is a target biomolecule.

9. The method of claim 8, wherein the target biomolecule is a polypeptide, protein, DNA, RNA or polysaccharide.

10. The method of any of the previous claims, wherein step (c) comprises forming a covalent bond linking the member of the first set and the member of the second set.

11. The method of any of the previous claims, wherein screening step (d) comprises a biological measurement.

12. The method of any of the previous claims, wherein a member of the first set and a member of the second set bind to the target in a 1:1 ratio.

13. The method of any of the previous claims, wherein further comprises assembling a third set of target binding ligands that compete for binding to the first binding site on the target and a fourth set of target binding ligands that compete for binding to the first binding site on the target, where members of each of the third set and the fourth set compete with members of the first set for binding to the first binding site, but members of the third set do not compete with members of the fourth set for binding to the target.

14. The method of claim 13 further comprises covalently linking at least one member of the third set or the fourth set and at least one member of the second set to provide a second set of linked ligands; and screening the second set of linked ligands to identify members thereof that bind to the target.

15. A method of preparing a drug lead compound that binds to a target, comprising covalently linking at least one member of a first set of target binding ligands that compete for non-covalent binding to a first binding site on the target and at least one member a second set of target binding ligands that compete for non-covalent binding to a second binding site on the target set to provide a first set of linked ligands.

16. The method of claim 15, wherein the first binding site is the same as the second binding site.

17. The method of claim 15 or 16, wherein the first binding site is not the same as the second binding site.

18. The method of any of claims 15-17, further comprises screening the set of linked ligands to identify members thereof that bind to the target.

19. The method of any of claims 15-18, further comprises covalently linking at least one member of a third set of target binding ligands that compete for binding to the first binding site on the target or at least one member of a fourth set of target binding ligands that compete for binding to the first binding site on the target to form a second set of linked ligands, where members of each of the third set and the fourth set compete with members of the first set for binding to the first binding site, but members of the third set do not compete with members of the fourth set for binding to the target.

20. A method for inhibiting the binding of a second biomolecule to a first biomolecule, comprising:
contacting the first and second biomolecules with a binding inhibitory amount of a compound identified according to any of claims 1-19, where the compound binds to the first biomolecule and inhibits the binding of the second biomolecule.
